(19) United States
(12) Patent Application Publication
(10) Pub. No.: US 2022/0308013 A1
(43) Pub. Date: Sep. 29, 2022

(54) METHOD FOR CORRECTING DATA RELATED TO ELECTROPHORESIS, METHOD FOR DETERMINING WHETHER PEAK IS SAMPLE-DERIVED PEAK OR SPIKE, APPARATUS, AND PROGRAM

(71) Applicant: HITACHI, LTD., Tokyo (JP)

(72) Inventors: Takeshi ISHIDA, Tokyo (JP); Yoshio KAMURA, Tokyo (JP); Yusuke GOTO, Tokyo (JP); Takahide YOKOI, Tokyo (JP)

(21) Appl. No.: 17/669,258

(22) Filed: Feb. 10, 2022

(30) Foreign Application Priority Data
Mar. 25, 2021 (JP) 2021-050981

(51) Int. Cl. G01N 27/447; G01N 27/416

(52) U.S. Cl. CPC G01N 27/44721; G01N 27/4163

(57) ABSTRACT

An electrophoresis data correction device or a post-color-call data correction device selects specific wavelength data from first data, performs filtering processing to cut some or all of components on a high frequency side, compares peak intensities of the specific wavelength data before and after the filtering processing for each cutoff frequency, calculates, as a first cutoff frequency, the minimum cutoff frequency at which a decrease in peak intensity of the specific wavelength data falls within a predetermined allowable range, and corrects the first data or post-color-call data of the first data by performing filtering processing with the first cutoff frequency.

### BACKGROUND OF THE INVENTION

### 1. Field of the Invention

The present invention relates to a method for correcting data by removing a part of a noise component from the data related to electrophoresis, a method for determining whether a peak in data related to electrophoresis is a sample-derived peak or a spike, an apparatus, and a program.

### 2. Description of the Related Art

With the development of the genome analysis technology, correlations between various diseases of human beings and gene mutations have been clarified. An acquired gene mutation derived from a disease such as cancer is characterized in that it is difficult to predict a mutation occurrence position on a genome and it is difficult to predict a mutation abundance ratio in an individual or a tissue. For example, a cancer tissue sample excised from a cancer patient contains cancer cells and normal cells and further, the cancer cells contain a variety of gene mutations, and thus, an abundance ratio of cells having a gene mutation in a specific position of a specific gene in the sample is sometimes extremely low. Therefore, a highly sensitive detection method is required in order to detect the acquired gene mutation derived from the disease. Further, there is also a case where not only the presence or absence of a gene mutation in a specific position of a target gene but also its abundance ratio is also taken as an index when selecting a therapeutic method or a therapeutic drug. Therefore, not only highly sensitive detection of a gene mutation but also quantification of its abundance ratio is important.

A conventional DNA sequencer using the Sanger's method is intended for determination of a base sequence, and thus, has problems that the detection power of a gene mutation that exists in a trace amount, that is, sensitivity is insufficient and that a range in which its abundance ratio can be quantified, that is, a dynamic range is narrow. Various optical systems have been proposed for an increase in sensitivity and an increase in dynamic range, and studies have also been conducted in terms of data processing. In particular, the increase in sensitivity and the increase in dynamic range by the data processing do not involve a change of an optical system, and thus, can be introduced at relatively low cost.

For example, WO 2015/015585 A presents a method for detecting a gene mutation with high sensitivity and quantifying the gene mutation with high precision by comparing a measured and calculated relative signal intensity of a nucleic acid sample with a relative signal intensity of a known nucleic acid sample stored in advance.

Further, WO 2016/132422 A discloses a method for estimating the magnitude of a noise component with high accuracy by performing time-frequency analysis on measurement data to acquire waveform data representing temporal changes of a plurality of frequency components, and analyzing the acquired waveform data.

### SUMMARY OF THE INVENTION

However, the conventional technology based on the data processing has a problem that it is necessary to construct a database in advance.

Although the method in WO 2015/015585 A is an effective and excellent method, it is necessary to construct a known information database in advance in order to perform such a comparison with known information. Since there is a variety of gene mutations, a relatively large database is required, and further, periodic data expansion is required to cope with new target genes.

Note that the method in WO 2016/132422 A is an excellent method for grasping a noise level, and leads to an increase in sensitivity and an increase in dynamic range if an application to removal of a noise component is possible, but there is no mention regarding a guideline, a method, and an effect of the noise component removal.

The present invention has been made to solve the above problems, and an object thereof is to provide a technology for achieving an increase in sensitivity or an increase in dynamic range by data processing while eliminating the need for constructing a database in advance.

An example of a method according to the invention is a method for correcting data related to electrophoresis by removing a part of a noise component from the data, and includes: acquiring first data by performing electrophoresis of a labeled nucleic acid sample to be analyzed and simultaneously detecting label signals at a plurality of measurement wavelengths, the first data being detection intensity waveform data containing a sample-derived component and a noise component; selecting, from the first data, specific wavelength data corresponding to one or more measurement wavelengths which is a target of time-frequency analysis; performing filtering processing to cut some or all of components on a high frequency side on the specific wavelength data for one or more cutoff frequencies; comparing peak intensities of the specific wavelength data before and after the filtering processing for each of the cutoff frequencies; calculating, as a first cutoff frequency, a minimum cutoff frequency at which a decrease in peak intensity of the specific wavelength data falls within a predetermined allowable range among the cutoff frequencies; and correcting the first data or post-color-call data of the first data by performing filtering processing with the first cutoff frequency.

Further, an example of a method according to the present invention is a method for determining whether each of peaks in data related to electrophoresis is a sample-derived peak or a spike, and includes: performing correction using the above-described method; calculating a peak intensity change rate for each of the peaks based on a peak intensity before the correction and a peak intensity after the correction; and determining that the peak at which an absolute value of the peak intensity change rate is greater than a predetermined threshold at one or more measurement wavelengths is the spike.

According to the technology of the present invention, it is possible to achieve the increase in sensitivity or the increase in dynamic range by the data processing while eliminating the need for constructing the database in advance.

For example, a large-scale database is not required and an optical system is not changed, and thus, introduction at low cost can be achieved.

Another characteristic relating to the present invention will become apparent from the description of the present specification and the accompanying drawings. Further, other objects, configurations, and effects will be apparent from the following description of embodiments.

### BRIEF DESCRIPTION OF THE DRAWINGS

FIG. 1 is a configuration diagram of an electrophoresis data correction device according to a first embodiment of the present invention;

FIG. 2 is a flowchart of an electrophoresis data correction method according to the first embodiment;

FIG. 3 illustrates examples of waveforms of electrophoresis data in a case with a sample and in a case without a sample;

FIG. 4 illustrates power spectra of the waveforms of FIG. 3;

FIG. 5 is a graph obtained by making the horizontal axis of the power spectra of FIG. 4 linear;

FIG. 6 illustrates power spectra before and after smoothing processing;

FIG. 7A illustrates an example of a waveform of electrophoresis data including relatively small spikes;

FIG. 7B illustrates a power spectrum of the waveform of FIG. 7A;

FIG. 7C illustrates an example of a waveform of electrophoresis data including relatively large spikes;

FIG. 7D illustrates a power spectrum of the waveform of FIG. 7C;

FIG. 8A illustrates an example of a waveform of electrophoresis data;

FIG. 8B illustrates a power spectrum of the waveform of FIG. 8A;

FIG. 9 illustrates examples of a change in intensity, a change in noise, and a change in dynamic range of a sample-derived peak component before and after filtering processing with respect to a cutoff frequency of a low-pass filter;

FIG. 10A is an enlarged view of the waveform of FIG. 8A;

FIG. 10B is a graph obtained by correcting the waveform of the electrophoresis data of FIG. 10A;

FIG. 11 illustrates a flowchart illustrating a processing example of step S6 in FIG. 2;

FIG. 12 illustrates a flowchart of an electrophoresis data correction method according to a second embodiment of the present invention;

FIG. 13A illustrates a waveform of post-color-call data obtained using electrophoresis data that is not corrected;

FIG. 13B is an enlarged view of the waveform of the post-color-call data of FIG. 13A;

FIG. 13C illustrates a waveform of post-color-call data obtained using corrected electrophoresis data;

FIG. 13D is an enlarged view of the waveform of the post-color-call data of FIG. 13C;

FIG. 14A illustrates a waveform of post-color-call data in a case where correction is performed on the post-color-call data without performing correction on electrophoresis data;

FIG. 14B is an enlarged view of the waveform of FIG. 14A;

FIG. 15 is a configuration diagram of a post-color-call data correction device according to a third embodiment of the present invention;

FIG. 16 is a flowchart of a post-color-call data correction method according to the third embodiment;

FIG. 17 is a flowchart of a post-color-call data correction method according to a fourth embodiment of the present invention;

FIG. 18A illustrates a waveform of electrophoresis data not including a spike;

FIG. 18B is a graph obtained by correcting the waveform of the electrophoresis data of FIG. 18A;

FIG. 18C illustrates a waveform of electrophoresis data including a spike having a peak value saturated at a measurement upper limit value;

FIG. 18D is a graph obtained by correcting the waveform of the electrophoresis data of FIG. 18C;

FIG. 18E illustrates a waveform of electrophoresis data including a relatively small spike;

FIG. 18F is a graph obtained by correcting the waveform of the electrophoresis data of FIG. 18E;

FIG. 18G illustrates a waveform of electrophoresis data including a relatively small spike having successive close values near peak values;

FIG. 18H is a graph obtained by correcting the waveform of the electrophoresis data of FIG. 18G;

FIG. 19A illustrates a waveform of electrophoresis data including a spike having a peak value saturated at a measurement upper limit value at three successive points.

FIG. 19B is a graph obtained by correcting the waveform of the electrophoresis data of FIG. 19A;

FIG. 20A is a graph obtained by removing the spike from the waveform of the electrophoresis data of FIG. 18G and complementing data points; and

FIG. 20B is a graph obtained by correcting the waveform of the electrophoresis data of FIG. 20A.

### DESCRIPTION OF PREFERRED EMBODIMENTS

Hereinafter, embodiments of the present invention will be described with reference to the drawings. Note that modes for carrying out the present invention are not limited to the embodiments to be described later, and various modifications can be made within the scope of the technical idea.

### (1) First Embodiment

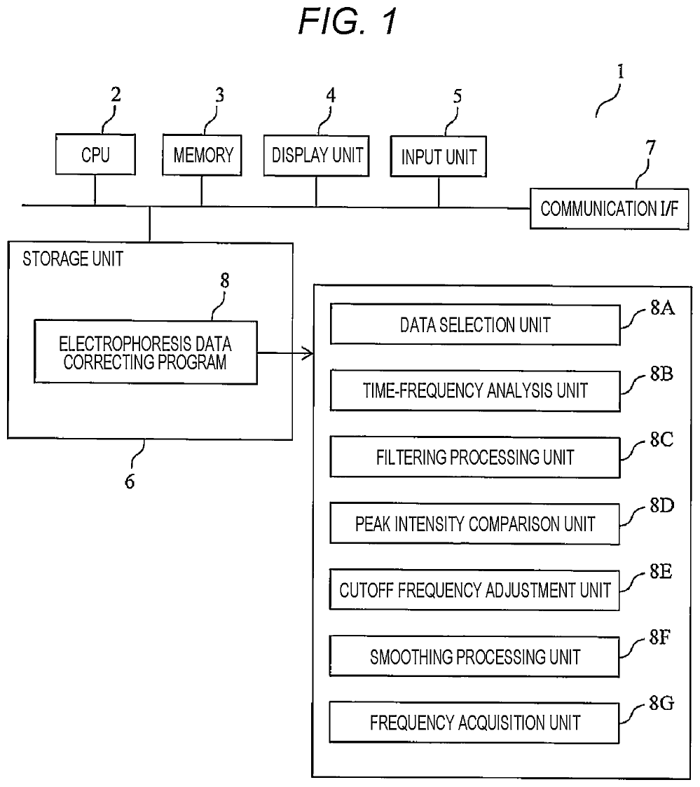

FIG. 1 illustrates a configuration of an electrophoresis data correction device 1 that corrects electrophoresis data according to the present embodiment. The electrophoresis data correction device 1 is, for example, a general-purpose computer, and includes a central processing unit (CPU) 2, a memory 3, a display unit 4 (for example, a monitor), an input unit 5, a storage unit 6 including a mass storage device such as a hard disk, and a communication interface 7.

The electrophoresis data correction device 1 is connected to a capillary electrophoresis sequencer (not illustrated) through the communication interface 7.

The storage unit 6 stores an operating system (OS) and an electrophoresis data correction program 8. When the CPU 2 executes the electrophoresis data correction program 8, the electrophoresis data correction device 1 functions as a data selection unit 8A, a time-frequency analysis unit 8B, a filtering processing unit 8C, a peak intensity comparison unit 8D, a cutoff frequency adjustment unit 8E, a smoothing processing unit 8F, and a frequency acquisition unit 8G which will be described later.

The electrophoresis data correction device 1 is configured to execute a method according to the present embodiment. Further, the electrophoresis data correction program 8 causes a computer to execute such a method, thereby causing the computer to function as the electrophoresis data correction device 1. In the present embodiment, a method for correcting data related to electrophoresis by removing a part of a noise component from the data is executed.

The method according to the present embodiment includes acquiring electrophoresis data (first data) by performing electrophoresis of a labeled nucleic acid sample to be analyzed and simultaneously detecting label signals at a plurality of measurement wavelengths. This data is detection intensity waveform data containing a sample-derived component and a noise component, and includes data at the plurality of wavelengths. In the present embodiment, this electrophoresis data is set as a correction target.

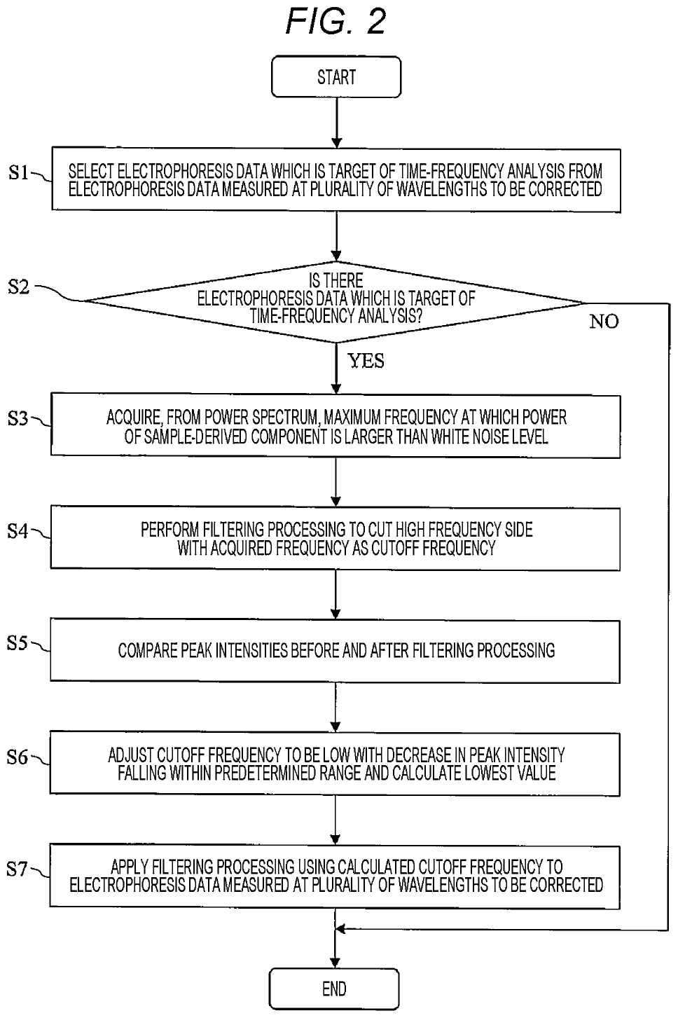

Hereinafter, an electrophoresis data correction method using the electrophoresis data correction device 1 of the present embodiment will be described with reference to a flowchart of FIG. 2. A process in FIG. 2 starts to be executed based on, for example, an execution instruction from a user.

First, data (specific wavelength data) corresponding to one or more measurement wavelengths which is a target of time-frequency analysis is selected from the electrophoresis data (step S1). The selection can be made, for example, based on a user's instruction. Further, the selection may be automatically performed by the electrophoresis data correction device 1 based on a predetermined criterion.

If there is no corresponding specific wavelength data (NO in step S2), the process in FIG. 2 is ended without performing analysis.

If the corresponding specific wavelength data exists (YES in step S2), the maximum frequency at which the power of the sample-derived component is higher than the power at a white noise level in a power spectrum of a specific wavelength is acquired (step S3). The frequency acquired here is used as an initial value of a cutoff frequency to be described later, and is referred to as an initial cutoff frequency, hereinafter. For example, the time-frequency analysis unit 8B acquires the power spectrum from the electrophoresis data, and the frequency acquisition unit 8G acquires the initial cutoff frequency.

Although the initial cutoff frequency can be arbitrarily set, the calculation amount of data correction processing can be reduced as will be described later if the initial cutoff frequency is set to the maximum frequency at which the power of the sample-derived component is higher than the power of the white noise level as described above.

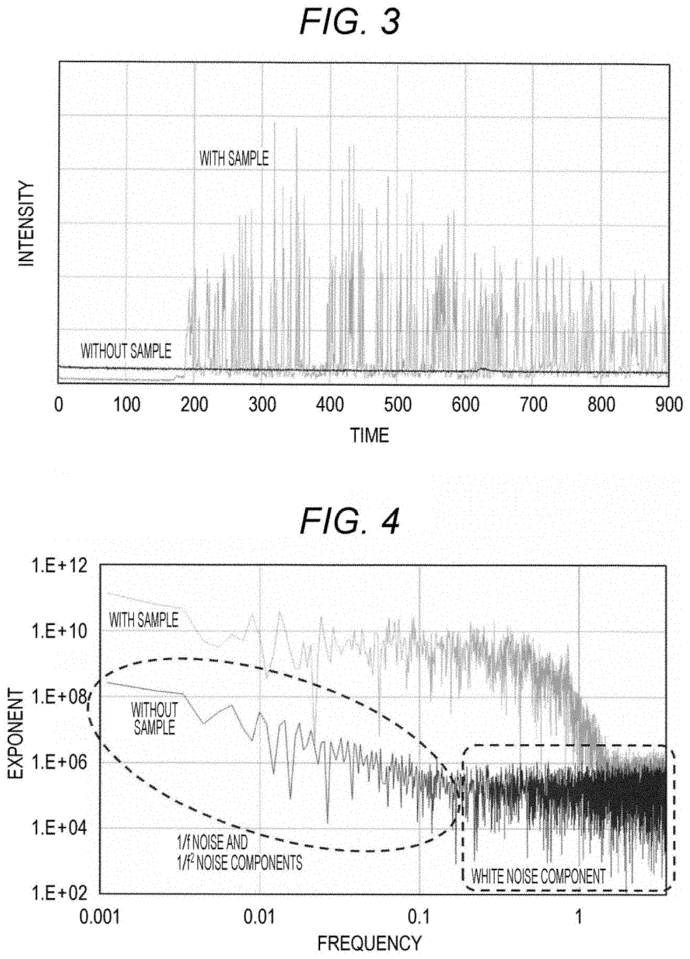

A detailed description of step S3 will be given first for convenience in order to describe the details of steps S1 to S2. FIG. 3 illustrates examples of electrophoresis data in a case (gray) with a sample and in a case (black) without a sample. The data with a sample shows a waveform including multiple sample-derived peaks. On the other hand, the data without a sample shows a substantially constant value.

FIG. 4 illustrates power spectra obtained as the time-frequency analysis unit 8B performs time-frequency analysis on each waveform data using Fourier transform. The power spectrum without a sample contains a white noise component, a 1/f noise component, and a 1/f² noise component. The noise components are derived from, for example, a photodetector constituting the capillary electrophoresis sequencer and a polymer in a capillary.

On the other hand, it can be seen that the power spectrum with a sample has white noise on a high frequency side, but has high power on a low frequency side of a certain frequency. This means that the power of the sample-derived component is distributed on the low frequency side of the certain frequency.

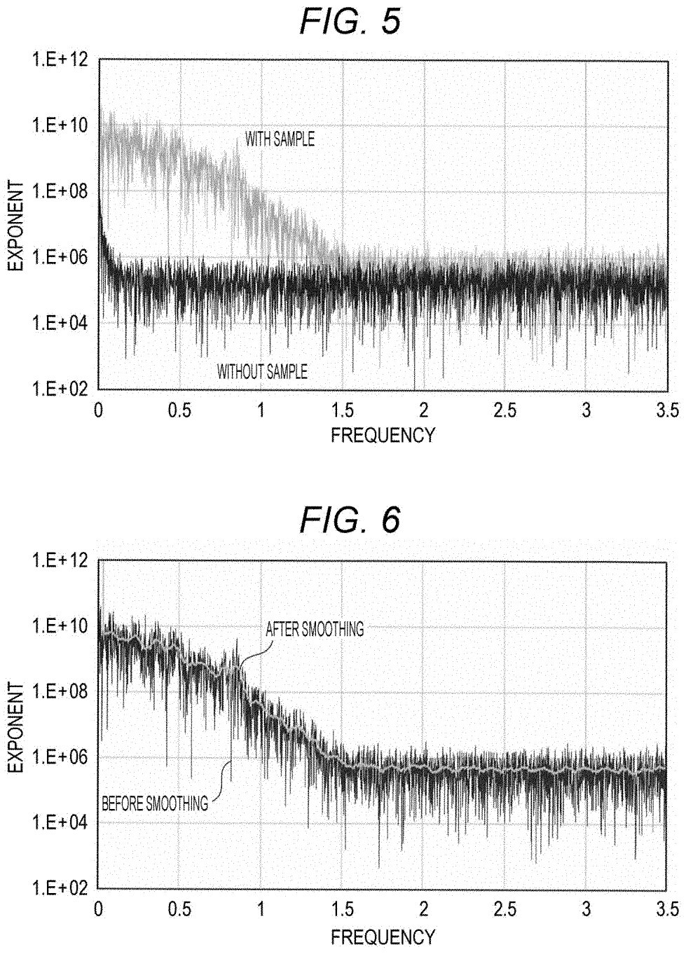

Since the horizontal axis of a graph of FIG. 4 is a logarithm, a graph obtained by making the horizontal axis linear is illustrated in FIG. 5. The power is almost constant at the white noise level on the high frequency side of a frequency of about 1.5 Hz in both the cases with and without a sample, whereas the power with a sample is high on the low frequency side of about 1.5 Hz.

Although FIGS. 3 to 5 illustrate the electrophoresis data without a sample and the power spectra thereof, the electrophoresis data without a sample is not necessarily required in order to acquire the initial cutoff frequency. The initial cutoff frequency at which the power of the sample-derived component is higher than the white noise level can be acquired based only on the electrophoresis data with a sample and the power spectrum thereof.

In step S3, the smoothing processing unit 8F may perform smoothing on the power spectrum. Examples of specific smoothing methods include a moving average method, an adjacent averaging method, a Savitzky-Golay method, an FFT filter, a percentile filter, LOWESS/LOESS smoothing, and the like. That is, the method according to the first embodiment may include performing smoothing processing on the power spectrum upon acquiring the initial cutoff frequency.

FIG. 6 illustrates results before and after smoothing by the adjacent averaging method at 51 points is applied to the power spectrum with the sample in FIG. 5. Data before smoothing is indicated by black, and data after smoothing is indicated by gray. Since the smoothing is performed, it is easy to acquire the initial cutoff frequency by threshold determination. For example, if the threshold is set to be twice an average value of components in a frequency range of 2.5 to 3.5 Hz, the maximum frequency at which the power of the sample-derived component is higher than the white noise level is 1.48 Hz.

Note that it is unnecessary to automatically calculate the initial cutoff frequency in step S3. For example, the user may read the maximum frequency at which the power of the sample-derived component is higher than the white noise level from the power spectrum or the smoothed power spectrum, and input the read maximum frequency to the frequency acquisition unit 8G.

In the power spectrum with a sample, there is a case where the maximum frequency at which the power of the sample-derived component is higher than the white noise level depends on the electrophoresis speed, and thus, depends on, for example, an electrophoresis voltage, the viscosity of the polymer, the temperature of the capillary, and the like among measurement conditions. Meanwhile, there is a case where the maximum frequency does not depend on a wavelength or a color of light to be observed.

However, there is a case where the magnitude of a sample-derived peak extremely differs depending on the wavelength or color of light to be observed. If the sample-derived peak is small, the sample-derived component is buried in white noise in the power spectrum so that it is difficult to acquire an appropriate initial cutoff frequency. Therefore, in step S1 described above, it is desirable to select electrophoresis data in which the sample-derived peak is sufficiently large.

The maximum frequency at which the power of the sample-derived component is higher than the white noise level in the power spectrum depends on measurement conditions. Therefore, prior to the start of the process in FIG. 2, an appropriate initial cutoff frequency for each of one or more representative measurement conditions may be acquired and held in the storage unit 6.

In a case where the initial cutoff frequency for the representative measurement condition has been acquired in advance and the data selected in step S1 is data measured under the representative measurement condition, the process may proceed to filtering processing (step S4) to be described later without performing the determination in step S2.

Further, the user may set an expected value of the maximum frequency at which the power of the sample-derived component is higher than the white noise level. In a case where the user sets the predicted value, the predicted value may be set as the initial cutoff frequency, and the process may proceed to the filtering processing (step S4) to be described later without performing the determination in step S2.

Step S3 has been described as above. Next, steps S1 to S2 will be described. In a DNA sequencer using the Sanger's method, a sharp peak, called a spike, in which a plurality of wavelengths and colors overlap each other due to mixed bubbles and foreign matters sometimes appears in a waveform of electrophoresis data even if a sample has not been migrated.

The spike is steep as compared with a sample-derived peak waveform and has a small number of data points forming a peak. A height of the peak is often extremely large, but is the same as a height of a sample-derived peak in some cases. It is necessary to distinguish between the spike and the sample-derived peak waveform during analysis such as sequence analysis or fragment analysis, so that various methods are used.

Specific examples of a method for determining a spike include determination methods respectively using a peak height, a half-value width, and a range of overlapping wavelengths or colors, and a method using a combination thereof.

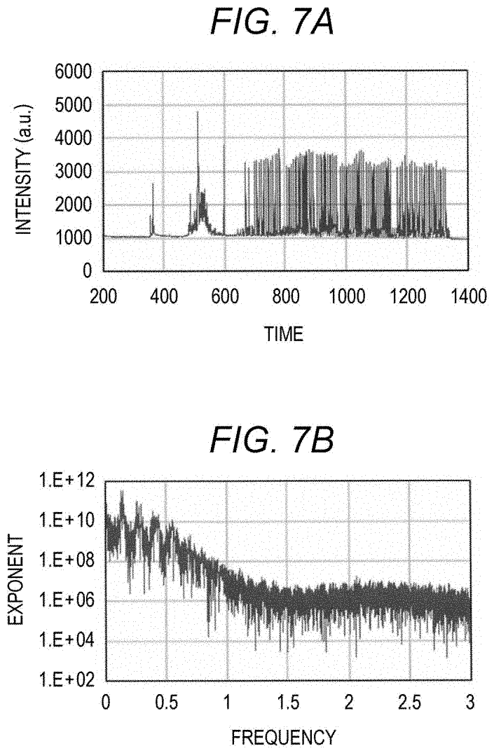

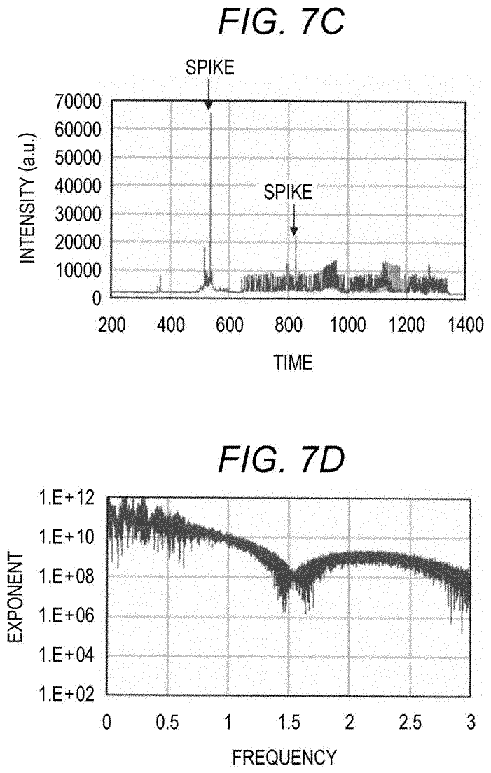

There is a case where it is difficult to acquire an appropriate initial cutoff frequency if electrophoresis data contains a large spike. FIG. 7A illustrates electrophoresis data including a relatively small spike, FIG. 7B illustrates a power spectrum thereof, FIG. 7C illustrates electrophoresis data including relatively large spikes, and FIG. 7D illustrates an example of a power spectrum thereof. The two pieces of electrophoresis data have been acquired for the same sample simultaneously at different wavelengths.

The spike exists near time 824 in the electrophoresis data of FIG. 7A, but has the same magnitude as a sample-derived peak, and thus, it is difficult to clearly confirm the spike in this drawing. In the power spectrum of the waveform of FIG. 7A illustrated in FIG. 7B, it can be confirmed that a high frequency side is at the white noise level and the power is high on a low frequency side, which is similar to the power spectrum with a sample of FIG. 5. Therefore, the initial cutoff frequency can be appropriately calculated.

On the other hand, in the electrophoresis data of FIG. 7C, a spike indicating a value saturated at a measurement upper limit value exists near time 536 and a spike higher than a sample-derived peak exists near time 824. The power spectrum of the waveform of FIG. 7C illustrated in FIG. 7D is completely different from the power spectrum of FIG. 7B, and there is no flat spectrum region indicating the white noise level. Thus, it is difficult to clearly identify the maximum frequency at which the power of the sample-derived component is higher than the white noise level, and it is difficult to appropriately calculate the initial cutoff frequency.

A spike has a sharp waveform, and thus, has power in a wide frequency band. Since a spike having a large peak height has high power, a power spectrum of a sample-derived component is buried with even a small number of spikes. On the other hand, in a case of a spike having the same magnitude as a sample-derived peak, a power spectrum of a sample-derived component is not buried with a power spectrum of a spike component since the number of spikes is usually sufficiently smaller than the number of sample-derived peaks.

As described above, the electrophoresis data includes the simultaneously measured data of the plurality of measurement wavelengths. Upon selecting the specific wavelength data from the electrophoresis data in step S1, the possibility that an appropriate initial cutoff frequency can be calculated increases by not selecting data of a measurement wavelength including a large spike (several times to several tens of times or more of a sample-derived peak) as illustrated in FIG. 7C but selecting data of a measurement wavelength in which there is no spike as illustrated in FIG. 7A or data of a measurement wavelength wherein a peak height is about the same as a sample-derived peak even if there is a spike.

Such a criterion can be appropriately determined by those skilled in the art based on known techniques and the like, and can be defined based on, for example, the peak height, the half-value width, whether or not peaks appear to overlap each other at a plurality of measurement wavelengths, a range of colors (measurement wavelengths) at which peaks appear, and the like as described above. Further, data may be automatically selected based on the defined criterion.

The present inventors have experimentally confirmed that the maximum frequency at which power of a sample-derived component is higher than a white noise level in a power spectrum does not change even in pieces of electrophoresis data measured at different wavelengths as long as the same sample is simultaneously measured under the same electrophoresis condition.

Therefore, in step S1, the data selection unit 8A can select data of a measurement wavelength at which it is determined that there is no spike based on a predetermined criterion from the electrophoresis data measured at the plurality of wavelengths, or can select data of a measurement wavelength at which it is determined that a peak value of a spike falls within the same range as a peak value of a sample-derived component based on a predetermined criterion.

In step S4, the filtering processing unit 8C performs the filtering processing using the initial cutoff frequency acquired as described above. The filtering processing is to cut off some or all of components on the high frequency side of the initial cutoff frequency, and can be performed using, for example, a low-pass filter, a band-pass filter, or a combination thereof.

Next, the peak intensity comparison unit 8D compares peak intensities before and after the filtering processing (step S5).

The cutoff frequency adjustment unit 8E changes a cutoff frequency from the initial cutoff frequency, and calculates a cutoff frequency (first cutoff frequency) that is the minimum frequency among cutoff frequencies at which a decrease in peak intensity due to the filtering processing falls within a predetermined allowable range (step S6).

In step S6, the filtering processing to cut some or all of components on the high frequency side of the specific wavelength data is performed for one or more cutoff frequencies. Then, the peak intensities of the specific wavelength data before and after the filtering processing are compared for each cutoff frequency. Furthermore, among these cutoff frequencies, the minimum cutoff frequency at which the decrease in peak intensity of the specific wavelength data falls within the predetermined allowable range is calculated as the first cutoff frequency.

In the present embodiment, an increase in peak intensity is determined to fall within the allowable range. However, as modifications, the increase in peak intensity may be determined to be out of the allowable range, or it may be determined whether the increase in peak intensity falls within the allowable range based on an increase rate (for example, by a comparison with a predetermined threshold).

In this manner, the cutoff frequency adjustment unit 8E sets the initial cutoff frequency as an initial value of the cutoff frequency, and calculates the first cutoff frequency by repeating the filtering processing while lowering the cutoff frequency. Therefore, if the initial cutoff frequency is set to the maximum frequency at which the power of the sample-derived component is higher than the power of the white noise level, it is possible to omit the operation in a high frequency band in which calculation is unnecessary, so that the calculation amount can be reduced.

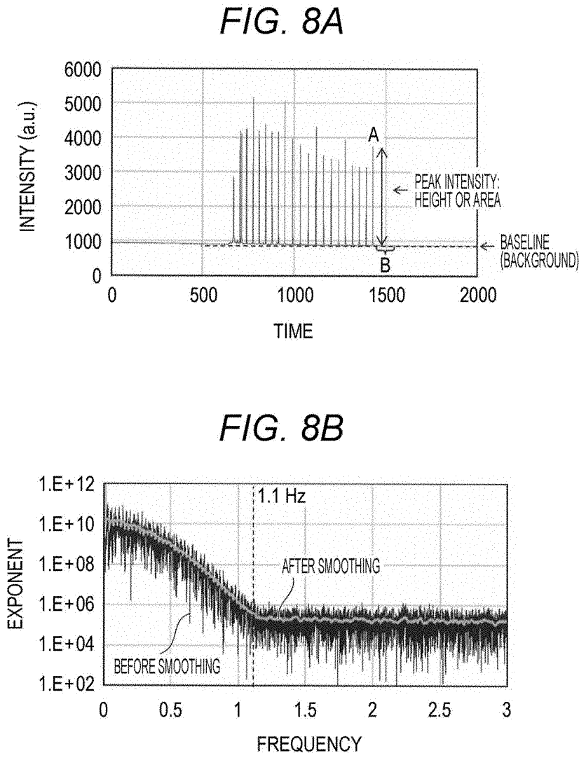

A case where electrophoresis data illustrated in FIG. 8A is set as a correction target will be described. FIG. 8B illustrates power spectrum obtained by analysis of the time-frequency analysis unit 8B. The initial cutoff frequency has been acquired as 1.1 Hz by smoothing of the smoothing processing unit 8F and threshold determination of the frequency acquisition unit 8G.

The filtering processing unit 8C applies a low-pass filter with the cutoff frequency of 1.1 Hz to the electrophoresis data illustrated in FIG. 8A, and the peak intensity comparison unit 8D compares peak intensities before and after the filtering processing. In the present embodiment, the peak intensity is represented using heights of all peaks in FIG. 8A, and this is compared before and after the filtering processing. For example, the height of peak A in FIG. 8A is represented using a difference between a peak top value and a background value (baseline). If the peak intensity is represented using the height of the peak in this manner, the peak intensity can be easily calculated.

In step S6, the filtering processing unit 8C may acquire the background value in the specific wavelength data. The background value can be appropriately acquired based on a known technique or the like. For example, the background value can be calculated as an average value of portions having no peak in the specific wavelength data.

The peak intensity may be represented using heights of some peaks instead of the heights of all the peaks. Further, the peak intensity may be represented not by the height of the peak but by an area of a peak. The area of the peak can be appropriately calculated based on a known technique or the like. For example, integration may be performed between times at which local minima or background values are given on both sides of a peak time, or a predetermined constant may be subtracted from a result of the integration. If the peak intensity is represented using the area of the peak, the intensity can be calculated in consideration of not only the value of the peak top but also the width.

A change in noise component according to cutoff frequencies will be described in order to describe effects of the present embodiment. An index of noise is a standard deviation of a portion having no sample-derived peak in electrophoresis data, and is compared before and after the filtering processing. In the example of FIG. 8A, a standard deviation of a time range B, that is, 500 data points centered on time 1500 is used as the index of noise.

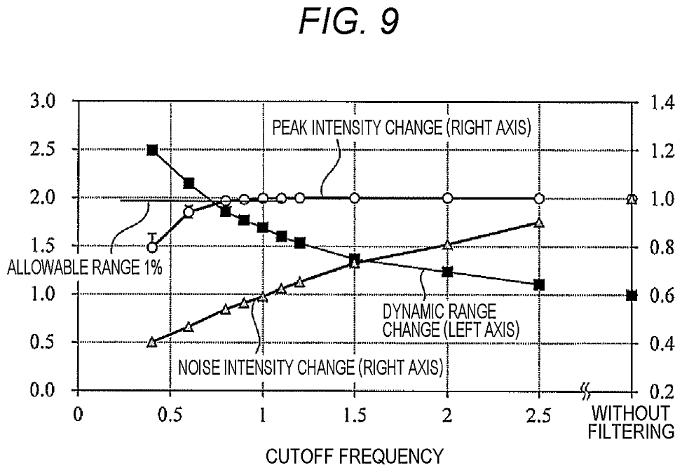

FIG. 9 illustrates a change in peak intensity, a change in noise intensity, and a change in dynamic range as changes before and after the filtering processing with respect to a cutoff frequency of a low-pass filter. A plot of the change in peak intensity is an average value regarding 22 sample-derived peaks illustrated in FIG. 8A. A change in each data is plotted by setting one as a value before the filtering processing, that is, wherein the filtering processing is not performed. An error bar is a standard deviation. For example, a case where the change in peak intensity is 0.9 means that the peak intensity has decreased by 10% before and after the filtering processing.

In the case where the low-pass filter with the cutoff frequency of 1.1 Hz was applied, the change in peak intensity was 0.998, the change in noise was 0.625, and the change in dynamic range was 1.599. This means that the peak intensity decreases by 0.2%, the noise decreases by 37.5%, and the dynamic range increases by 59.9%.

In the case where the allowable range of the decrease in peak intensity was set to 1% or less, it was calculated that the cutoff frequency could be lowered to 0.84 Hz based on interpolation. Note that FIG. 9 illustrates the allowable range of 1% to be wider than an actual allowable range for visibility. If a low-pass filter with a cutoff frequency of 0.84 Hz was applied, the change in peak intensity was 0.990, the change in noise was 0.549, and the change in dynamic range was 1.821. As the allowable range of the decrease in peak intensity is set to 1% or less in this manner, the noise can be greatly reduced without substantially decreasing the peak intensity, and the dynamic range can be greatly improved.

Note that an interpolation operation can be appropriately designed based on a known technique or the like. For example, a linear or non-linear interpolation operation can be performed according to the number of cutoff frequencies.

After step S6, the filtering processing unit 8C performs the filtering processing with the first cutoff frequency calculated as described above on the electrophoresis data (including a plurality of pieces of measurement wavelength data) to be corrected (step S7), thereby correcting the electrophoresis data.

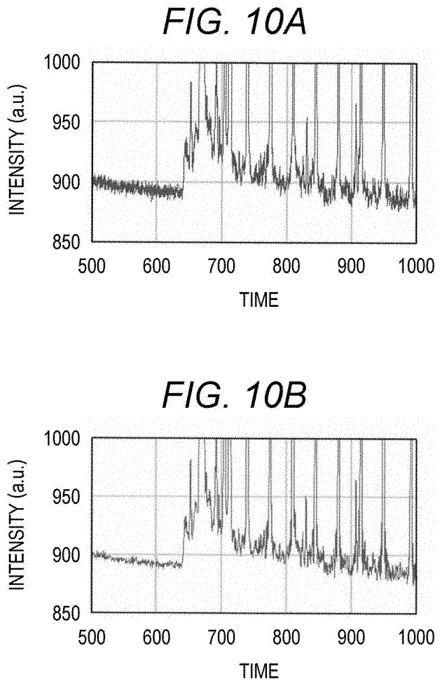

FIG. 10A illustrates an enlarged view of FIG. 8A as a waveform of the electrophoresis data before correction. FIG. 10B is a waveform of the electrophoresis data after correction. In a case of comparing FIGS. 10A and 10B, it can be confirmed that noise has been more reduced in FIG. 10B after correction.

In step S7, the user may be notified of the calculated first cutoff frequency through the display unit 4 such that the user can set a cutoff frequency to be used for correction. That is, the filtering processing unit 8C may perform the filtering processing based on the cutoff frequency set by the user, thereby correcting the electrophoresis data.

In this manner, it is possible to achieve an increase in sensitivity or an increase in dynamic range by data processing while eliminating the need for constructing a database in advance according to the first embodiment.

### (2) Processing Example of Step S6 in First Embodiment

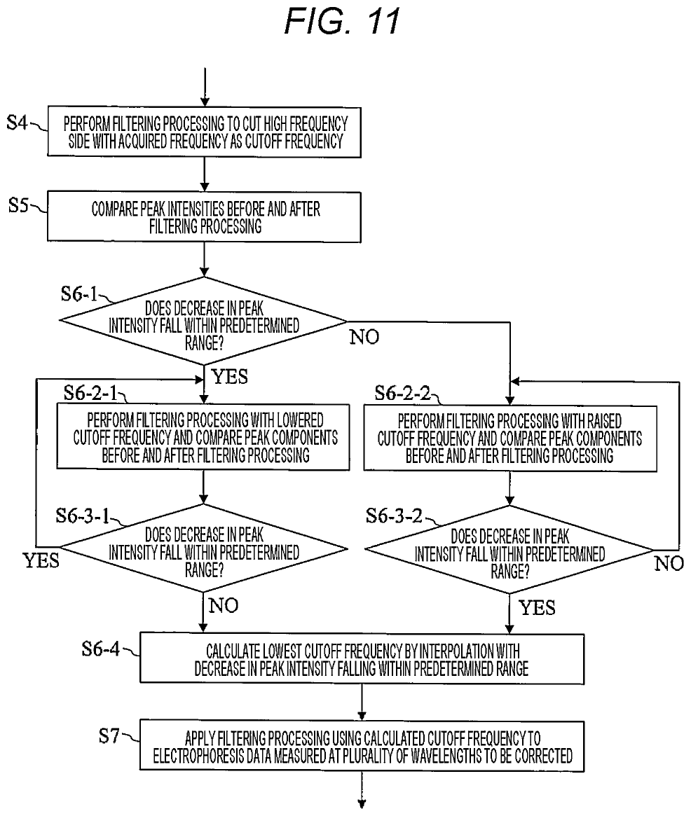

In steps S4 to S6 of FIG. 2, the filtering processing to cut components on the high frequency side is performed based on the acquired initial cutoff frequency, the peak intensities before and after the filtering processing are compared, and the cutoff frequency is adjusted to be low with the decrease in peak intensity falling within the predetermined allowable range to calculate the minimum value. A processing example of step S6 in such a process will be described more specifically with reference to a flowchart illustrated in FIG. 11.

FIG. 11 illustrates step S6 of FIG. 2 in more detail. In step S5, the peak intensity comparison unit 8D compares the peak intensities before and after the filtering processing, and then, determines whether the decrease in peak intensity falls within the allowable range (step S6-1).

If the decrease in peak intensity falls within the allowable range (YES in step S6-1), filtering processing with a lowered cutoff frequency is performed, and peak intensities before and after the filtering processing are compared (step S6-2-1). Here, whether a decrease in peak intensity falls within the allowable range is determined again (step S6-3-1).

If the decrease in peak intensity falls within the allowable range (YES), the process returns to step S6-2-1. If the decrease in peak intensity is out of the allowable range (NO), the minimum cutoff frequency (first cutoff frequency) at which the decrease in peak intensity falls within the allowable range is calculated by interpolation (step S6-4), and the process proceeds to step S7.

If the decrease in peak intensity is out of the allowable range in step S6-1 (NO in step S6-1), filtering processing with a raised cutoff frequency is performed, and peak intensities before and after the filtering processing are compared (step S6-2-2). Here, whether a decrease in peak intensity falls within the allowable range is determined again (step S6-3-2).

The process returns to step S6-2-2 if the decrease in peak intensity is out of the allowable range (NO). If the decrease in peak intensity falls within the allowable range (YES), the minimum cutoff frequency (first cutoff frequency) with the decrease in peak intensity falling within the allowable range is calculated by interpolation (step S6-4), and the process proceeds to step S7.

If an increase width and a decrease width of the cutoff frequency in steps S6-2-1 and S6-2-2 are set to 10% or less of the frequency acquired in step S3, the first cutoff frequency can be accurately calculated.

### (3) Second Embodiment

In the first embodiment described above in (1), in steps S4 to S6 of FIG. 2, the filtering processing to cut components on the high frequency side is performed using the acquired frequency as the cutoff frequency, the peak intensities before and after the filtering processing are compared, and the cutoff frequency is adjusted to be low with the decrease in peak intensity falling within the predetermined allowable range to calculate the minimum value. Upon adjusting the cutoff frequency to be low with the decrease in peak intensity falling within the allowable range, a flow of repeating performing filtering processing by raising or lowering the cutoff frequency and comparing peak intensities before and after the filtering processing is included.

In the present embodiment, however, filtering processing and peak intensity comparison are performed collectively to some extent, and a cutoff frequency at which a decrease in peak intensity due to the filtering processing becomes a predetermined value is calculated.

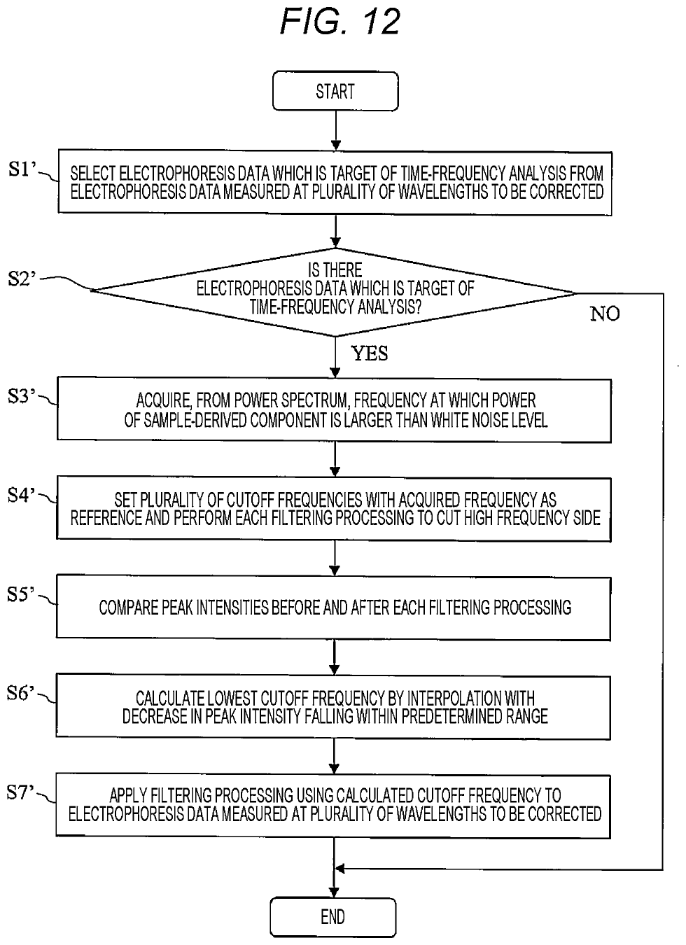

Hereinafter, an electrophoresis data correction method of the present embodiment will be described with reference to a flowchart of FIG. 12. Steps S1′ to S3′ are similar to steps S1 to S3 in FIG. 2 of the first embodiment described in (1).

A plurality of cutoff frequencies are set based on an initial cutoff frequency acquired in step S3′, and each filtering processing to cut components on the high frequency side is performed on electrophoresis data which is a target of time-frequency analysis (step S4′).

The plurality of cutoff frequencies may be set with a predetermined step size, for example, with the initial cutoff frequency as an upper limit.

Peak intensities before and after each filtering processing are compared (step S5′), and the minimum cutoff frequency (first cutoff frequency) with a decrease in peak intensity falling within an allowable range is calculated by interpolation (step S6′). Thereafter, filtering processing with the calculated first cutoff frequency is applied to electrophoresis data measured at a plurality of wavelengths to be corrected (step S7′), whereby the correction of the electrophoresis data ends.

In step S7, a user may be notified of the calculated first cutoff frequency such that the user can set a cutoff frequency to be used for correction. That is, the filtering processing unit 8C may perform the filtering processing based on the cutoff frequency set by the user, thereby correcting the electrophoresis data.

The first cutoff frequency can be accurately calculated if the step size of the frequency is set to 10% or less of the initial cutoff frequency upon setting the plurality of cutoff frequencies based on the initial cutoff frequency acquired in step S3′.

In this manner, it is possible to achieve an increase in sensitivity or an increase in dynamic range by data processing while eliminating the need for constructing a database in advance according to the second embodiment, which is similar to the first embodiment.

### (4) Third Embodiment

The electrophoresis data is corrected in the first embodiment described in (1) and the second embodiment described in (3). That is, data to be corrected is measurement value data (first data) obtained by electrophoresis, and this data is corrected by performing the filtering processing with the first cutoff frequency.

In a third embodiment, data after color call is corrected. That is, a method according to the third embodiment includes correcting the post-color-call data for the measurement value data (first data) obtained by electrophoresis by performing filtering processing with a first cutoff frequency.

The color call will be described. By performing electrophoresis for fluorescent dyes, a matrix that is information indicating fluorescence spectra of the respective fluorescent dyes used in a reagent kit is obtained. Based on this matrix, electrophoresis data, which is data of a signal spectrum for each wavelength band, can be converted into data of a signal spectrum for each type of fluorescent dye (post-color-call data). The post-color-call data also includes data at a plurality of wavelengths.

The color call is processing of acquiring signal spectrum data for each type of fluorescent dye used as a label. The color call can be performed, for example, by weighting data of measurement wavelengths of the electrophoresis data according to respective measurement wavelengths. Weighting factors for the measurement wavelengths vary depending on the type of fluorescent dye.

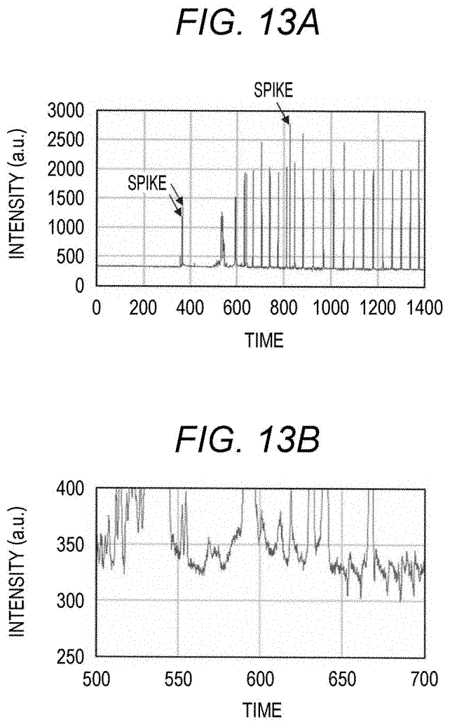

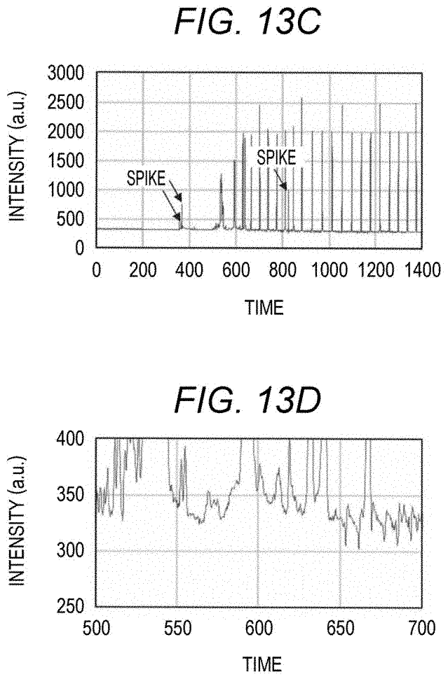

First, a description will be given with reference to FIGS. 13A to 13D regarding a noise reduction effect maintained even in post-color-call data if the correction according to the first or second embodiment is performed on electrophoresis data.

FIG. 13A illustrates post-color-call data obtained using electrophoresis data that is not corrected, and FIG. 13C illustrates post-color-call data obtained using corrected electrophoresis data obtained by performing the correction according to the first or second embodiment on the same electrophoresis data. FIGS. 13B and 13D are partially enlarged views of FIGS. 13A and 13C, respectively.

In a case of comparing FIGS. 13A and 13C, a difference in waveform other than spikes can hardly be confirmed. A reason why specific peaks are determined as the spikes is that sharp peaks overlapping at a plurality of wavelengths and saturated at a measurement upper limit value at the same time were observed in the electrophoresis data.

A decrease in height of the spike by the correction according to the first or second embodiment will be described later.

In a case of comparing FIGS. 13B and 13D, it can be confirmed that noise has been more reduced in FIG. 13D using the corrected electrophoresis data.

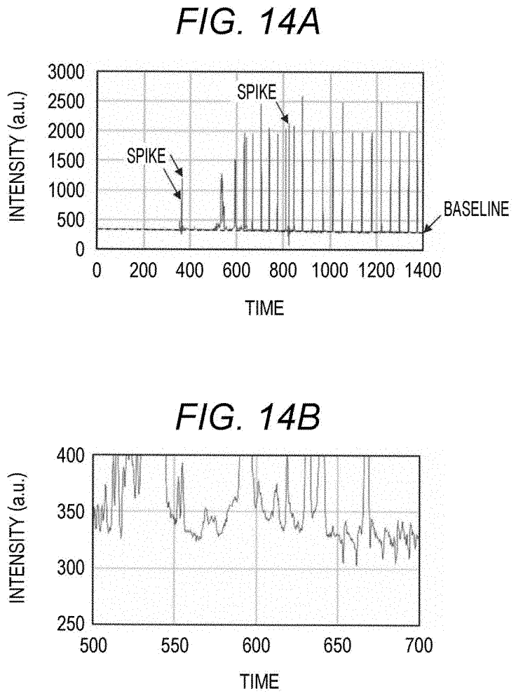

Next, FIG. 14A illustrates post-color-call data in a case where correction is performed on the post-color-call data without performing correction on the electrophoresis data, and FIG. 14B illustrates a partially enlarged view thereof. Conditions of filtering processing for the correction are the same as conditions of filtering processing performed in FIGS. 13C and 13D. Note that an initial cutoff frequency and a first cutoff frequency are calculated based on the electrophoresis data in the present embodiment.

In a case of comparing FIGS. 14A and 13A, a difference in waveform other than spikes can hardly be confirmed. In FIG. 14A, the height of the spike has decreased, and the bottom of the spike takes a value below a baseline. In a case of comparing FIGS. 14B and 13B, noise has been more reduced in FIG. 14B in which the post-color-call data is corrected. It can be seen that the noise reduction effects are equivalent in a case of comparing FIGS. 14B and 13D.

From the above, it can be said that noise of the post-color-call data can be reduced by correcting the post-color-call data.

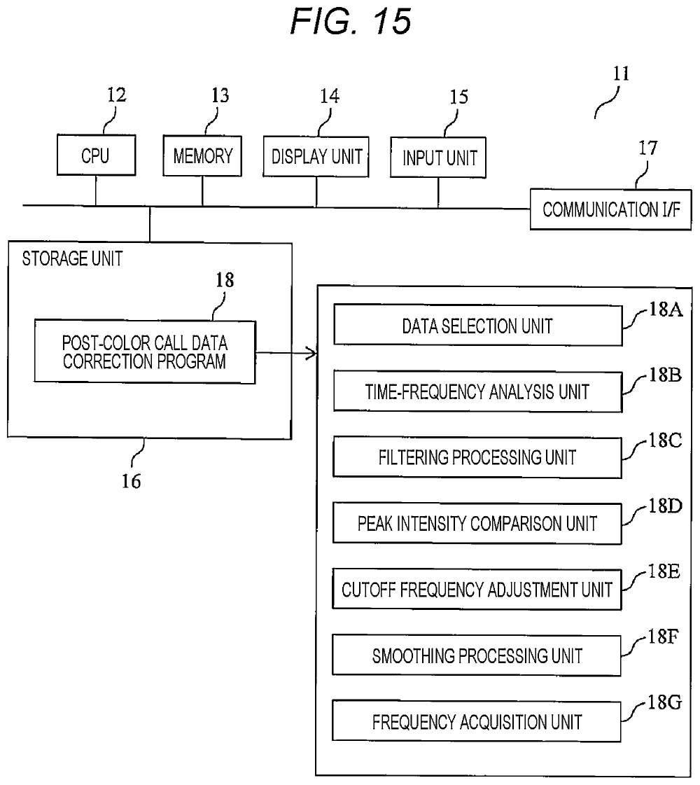

FIG. 15 illustrates a configuration of a post-color-call data correction device 11 that corrects the post-color-call data according to the present embodiment. The entity of the post-color-call data correction device 11 is a general-purpose personal computer, and includes a CPU 12 (central processing unit), a memory 13, a display unit 14 (for example, a monitor), an input unit 15, a storage unit 16 including a mass storage device such as a hard disk, and a communication interface 17.

The post-color-call data correction device 11 is connected to a capillary electrophoresis sequencer (not illustrated) through the communication interface 17.

The storage unit 16 stores an operating system (OS) and a post-color-call data correction program 18. When the CPU 12 executes the post-color-call data correction program 18, the post-color-call data correction device 11 functions as a data selection unit 18A, a time-frequency analysis unit 18B, a filtering processing unit 18C, a peak intensity comparison unit 18D, a cutoff frequency adjustment unit 18E, a smoothing processing unit 18F, and a frequency acquisition unit 18G which will be described later.

The post-color-call data correction device 11 is configured to execute the method according to the present embodiment. Further, the post-color-call data correction program 18 causes a computer to execute such a method, thereby causing the computer to function as the post-color-call data correction device 11.

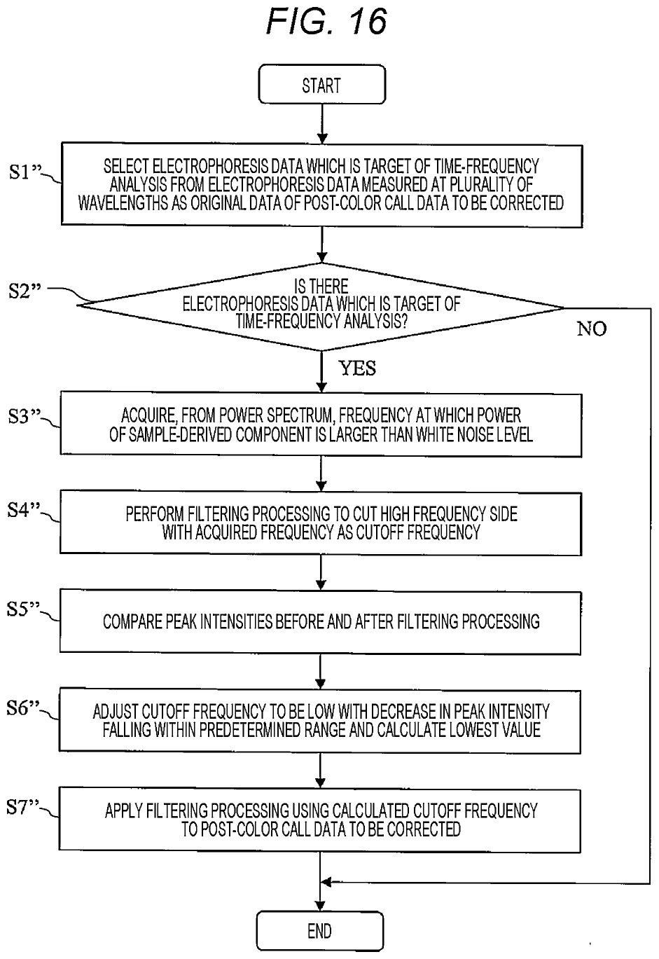

Hereinafter, a post-color-call data correction method using the post-color-call data correction device 11 will be described with reference to a flowchart of FIG. 16. A process in FIG. 16 starts to be executed based on, for example, an execution instruction from a user.

First, data (specific wavelength data) corresponding to one or more measurement wavelengths which is a target of time-frequency analysis is selected from electrophoresis data (step S1″). This electrophoresis data is original data of post-color-call data to be corrected. The selection can be made, for example, based on a user's instruction. Further, the selection may be automatically performed by the post-color-call data correction device 11 based on a predetermined criterion.

Subsequent steps S2″ to S6″ are similar to steps S2 to S6 in the flowchart of FIG. 2 of the first embodiment described in (1). Regarding a device configuration, the post-color-call data correction device 11 (FIG. 15) can be obtained by replacing the electrophoresis data correction program 8 constituting the electrophoresis data correction device 1 of FIG. 1 with the post-color-call data correction program 18.

In steps S2″ to S6″, constituent elements indicated by reference signs 12 to 18 and 18A to 18G in FIG. 15 perform similar operations as the constituent elements indicated by reference signs 2 to 8 and 8A to 8G in the first embodiment (FIG. 1) described in (1).

The filtering processing unit 18C applies filtering processing using the first cutoff frequency calculated in step S6″ to post-color-call data to be corrected (step S7″), whereby the correction of the post-color-call data ends.

In step S7″, the user may be notified of the calculated first cutoff frequency through the display unit 14 such that the user can set a cutoff frequency to be used for correction. That is, the filtering processing unit 8C may perform the filtering processing based on the cutoff frequency set by the user, thereby correcting the post-color-call data.

In this manner, it is possible to achieve an increase in sensitivity or an increase in dynamic range by data processing while eliminating the need for constructing a database in advance according to the third embodiment, which is similar to the first and second embodiments.

### (5) Fourth Embodiment

In the third embodiment described in (4), the correction target is the post-color-call data, but the first cutoff frequency is calculated using the electrophoresis data that is the original data thereof. In a fourth embodiment, a first cutoff frequency is calculated using post-color-call data as first data, instead of electrophoresis data, to correct the post-color-call data.

That is, in the present embodiment, the first data is the post-color-call data of measurement value data obtained by electrophoresis, and a method according to the present embodiment includes correcting the post-color-call data by performing filtering processing with the first cutoff frequency. Note that the post-color-call data is detection intensity waveform data containing a sample-derived component and a noise component, which is similar to the measurement value data.

The post-color-call data correction device 11 can have the same configuration as that of the third embodiment (FIG. 15) described in (4).

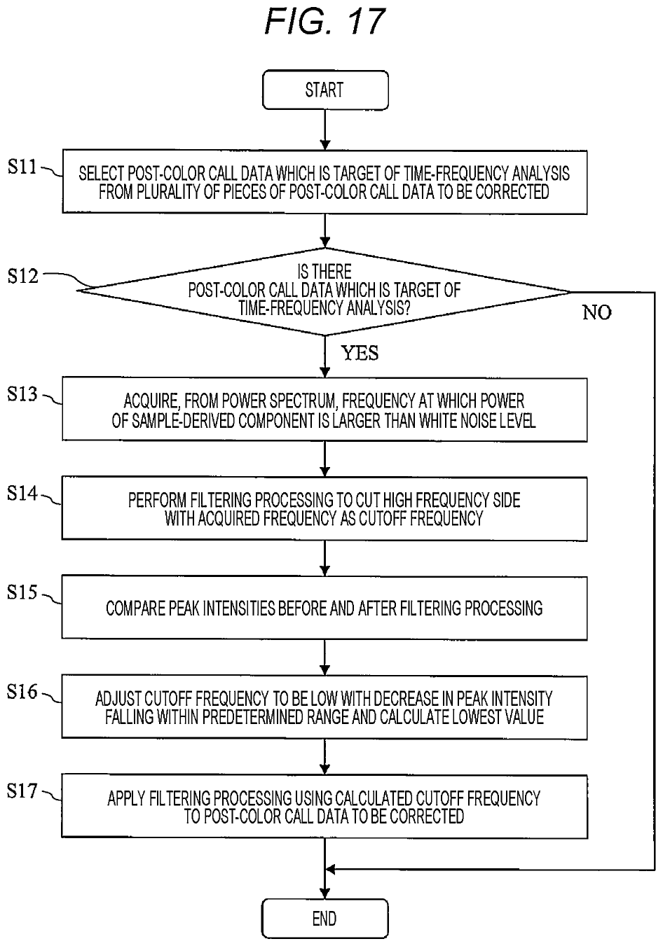

Hereinafter, a post-color-call data correction method according to the present embodiment will be described with reference to a flowchart of FIG. 17. A process in FIG. 17 starts to be executed based on, for example, an execution instruction from a user.

First, data (specific wavelength data) corresponding to one or more measurement wavelengths which is a target of time-frequency analysis is selected from the post-color-call data (step S11). The selection can be made, for example, based on a user's instruction. Further, the selection may be automatically performed by the post-color-call data correction device 11 based on a predetermined criterion.

The data selection unit 18A can select data of a measurement wavelength at which it is determined that there is no spike based on a predetermined criterion from the post-color-call data including data of a plurality of wavelengths, or can select data of a measurement wavelength at which it is determined that a peak value of a spike falls within the same range as a peak value of a sample-derived component based on a predetermined criterion.

If there is no corresponding specific wavelength data (NO in step S12), the process ends without performing analysis.

If the corresponding specific wavelength data exists (YES in step S12), the time-frequency analysis unit 18B acquires a power spectrum from the specific wavelength data, and the frequency acquisition unit 18G acquires, from the power spectrum, the maximum frequency (initial cutoff frequency) at which the power of the sample-derived component is higher than a white noise level (step S13).

Upon acquiring the initial cutoff frequency by the frequency acquisition unit 18G, the smoothing processing unit 18F may smooth the power spectrum.

Further, the user may read the maximum frequency at which the power of the sample-derived component is higher than the white noise level from the power spectrum or the smoothed power spectrum and input a value of the initial cutoff frequency. The frequency acquisition unit 18G may acquire this value.

The initial cutoff frequency depends on measurement conditions of electrophoresis data which is original data. Therefore, prior to the start of the process in FIG. 17, an appropriate initial cutoff frequency for each of one or more representative measurement conditions may be acquired and held in the storage unit 16.

In a case where the initial cutoff frequency for the representative measurement condition has been acquired in advance and the original electrophoresis data is data measured under the representative measurement condition, the process may proceed to filtering processing (step S14) to be described later without performing the determination in step S12.

Note that the initial cutoff frequency for the representative measurement condition may be acquired from the power spectrum of the post-color-call data, or may be acquired from a power spectrum of the electrophoresis data which is the original data.

Further, the user may set an expected value of the maximum frequency at which the power of the sample-derived component is higher than the white noise level. In a case where the user sets the predicted value, the predicted value may be set as the initial cutoff frequency, and the process may proceed to the filtering processing (step S14) to be described later without performing the determination in step S12.

In step S14, the filtering processing unit 18C performs the filtering processing using the initial cutoff frequency acquired as described above. The filtering processing is to cut off some or all of components on the high frequency side of the initial cutoff frequency, and can be performed using, for example, a low-pass filter, a band-pass filter, or a combination thereof.

Next, the peak intensity comparison unit 18D compares peak components before and after the filtering processing (step S15).

The cutoff frequency adjustment unit 18E changes a cutoff frequency from the initial cutoff frequency, and calculates a cutoff frequency (first cutoff frequency) that is the minimum frequency among cutoff frequencies at which a decrease in peak intensity due to the filtering processing falls within a predetermined allowable range (step S16).

The filtering processing unit 18C applies filtering processing using the calculated cutoff frequency to post-color-call data to be corrected (step S17), whereby the correction of the post-color-call data ends.

In step S17, the user may be notified of the calculated first cutoff frequency through the display unit 14 such that the user can set a cutoff frequency to be used for correction. That is, the filtering processing unit 18C may perform the filtering processing based on the cutoff frequency set by the user, thereby correcting the electrophoresis data.

In this manner, it is possible to achieve an increase in sensitivity or an increase in dynamic range by data processing while eliminating the need for constructing a database in advance according to the fourth embodiment, which is similar to the first to third embodiments.

### (6) Fifth Embodiment

In a fifth embodiment, spike determination using correction of electrophoresis data and post-color-call data is performed. That is, a method according to the fifth embodiment is a method for determining whether a peak in data related to electrophoresis is a sample-derived peak or a spike.

In the first to fourth embodiments, it has been described that a sharp peak, called a spike, in which a plurality of wavelengths and colors overlap each other due to mixed bubbles and foreign matters sometimes appears in the electrophoresis data even if a sample has not been migrated.

It is necessary to distinguish between the spike and a sample-derived peak waveform during analysis such as sequence analysis or fragment analysis, so that various methods are used. Specific examples of a method for determining a spike include determination methods respectively using a peak height, a half-value width, and a range of overlapping wavelengths or colors, and a method using a combination thereof.

However, a peak size, the half-value width, and the range of overlapping wavelengths or colors are different for each spike, and thus, a spike whose peak size is close to a sample-derived peak is sometimes erroneously determined as the sample-derived peak.

The spike can be determined with high accuracy by using the correction of the electrophoresis data and the post-color-call data described in the first to fourth embodiments. Hereinafter, the spike determination using the correction of the electrophoresis data will be described with an example.

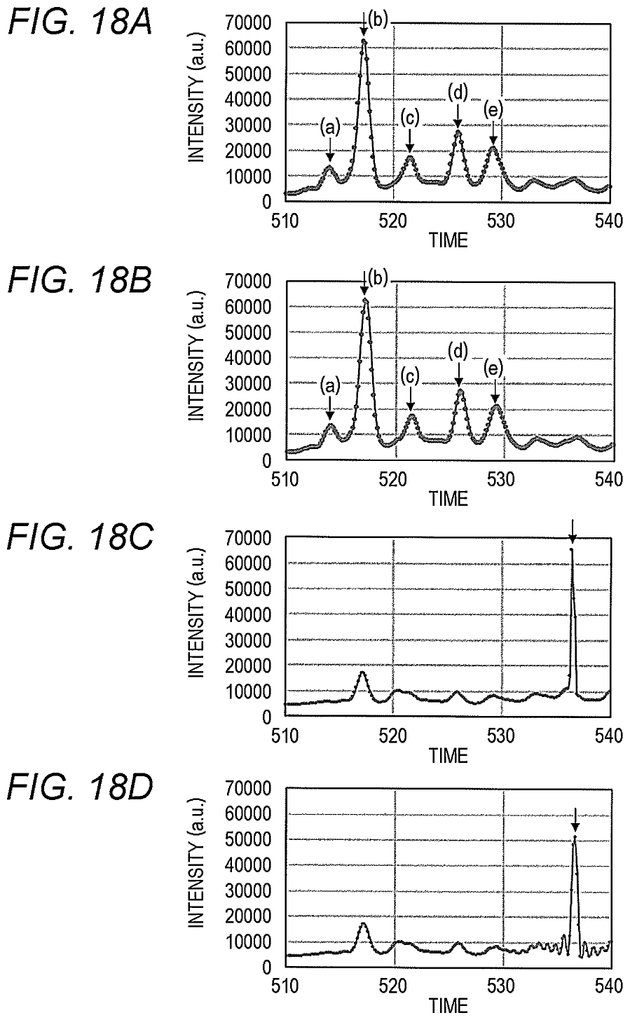

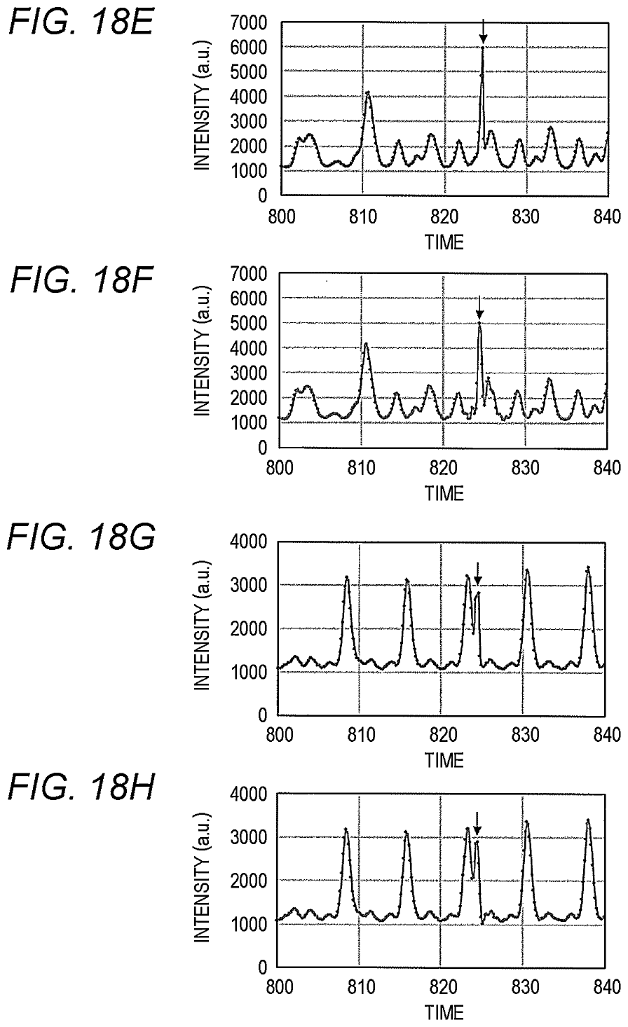

For cases wherein the electrophoresis data is corrected by setting an allowable range of a decrease in peak intensity to 1% or less, peak waveforms before the correction are illustrated in FIGS. 18A, 18C, 18E, and 18G, and the respective corrected peak waveforms are illustrated in FIGS. 18B, 18D, 18F, and 18H.

Arrows (a) to (e) in FIGS. 18A and 18B indicate sample-derived peaks. Change rates of peak heights due to the data correction were −0.63%, +0.06%, −0.36%, −0.61%, and −0.21%, respectively. In any case, the peak height decreases by 1% or less.

An arrow in FIG. 18C indicates a spike before the data correction. The maximum value is saturated at a measurement upper limit value. In a spike after the data correction, a peak height decreases as illustrated in FIG. 18D, and further, a waveform corresponding to a cutoff frequency appears at the bottom of the spike. A change rate of the peak height by the data correction was −22.6%.

An arrow in FIG. 18E indicates a spike before the data correction. The spike is relatively small, and a peak height is about the same as that of a sample-derived peak. In a spike after the data correction, a peak height decreases as illustrated in FIG. 18F, and further, a waveform corresponding to a cutoff frequency is added to the bottom of the spike and is slightly disturbed. A change rate of the peak height by the data correction was −18.8%.

An arrow in FIG. 18G indicates a spike before the data correction. The spike is relatively small, and a peak height is about the same as that of a sample-derived peak. The spike has successive close values near peak values. In a spike after the data correction, a peak height increases as illustrated in FIG. 18H, and further, a waveform corresponding to a cutoff frequency is added to the bottom of the spike and is slightly disturbed. A change rate of the peak height by the data correction was +4.0%.

As described above, a peak height decreases by the correction in most of the sample-derived peaks, but a change rate thereof is 1% or less, which is the same as a predetermined range of a decrease in intensity of a sample-derived peak component. The height of the sample-derived peak sometimes increases by the correction, but a change rate thereof is also 1% or less since the change rate is smaller than that in the case of the decrease.

On the other hand, the peak height decreases by the correction in most of the spikes, but the change rate thereof is 10% or more, which is larger than that of the sample-derived peak. Further, the peak height of the spike sometimes increases by the correction, but the change rate thereof is higher than that of the sample-derived peak even in the case of the increase.

Therefore, it is possible to determine whether the peak is the sample-derived peak or the spike based on a change rate of a peak intensity caused by the correction. For example, first, the peak intensity change rate is calculated for each peak based on a peak intensity before correction and a peak intensity after correction. Then, a peak at which an absolute value of the peak intensity change rate is greater than a predetermined threshold at one or more measurement wavelengths can be determined to be the spike, and a peak at which the absolute value of the peak intensity change rate is not greater than the predetermined threshold can be determined to be the sample-derived peak.

Although the height of the peak is used as an index of the peak intensity in the present embodiment, an area of the peak may be used as the index of the peak intensity.

Hereinafter, a description will be given using the height of the peak as the index of the peak intensity. The sample-derived peak and the spike can be discriminated except for a specific spike to be described later by determining, for example, a case where the absolute value of the peak height change rate caused by the correction is twice or more the allowable range (for example, 1%) used in step S6 as the spike. In this case, assuming that the allowable range is 1% or less, a case where the absolute value of the peak height change rate caused by the correction is 2% or more is determined as the spike.

Such a threshold can be set to an arbitrary value, but most of sample-derived peaks can be correctly determined as the sample-derived peaks if the threshold is set to a value exceeding an upper limit of the allowable range of step S6 (a value higher than 1% in the above example). If the threshold is twice or more the upper limit of the allowable range of step S6, more sample-derived peaks can be correctly determined as the sample-derived peaks.

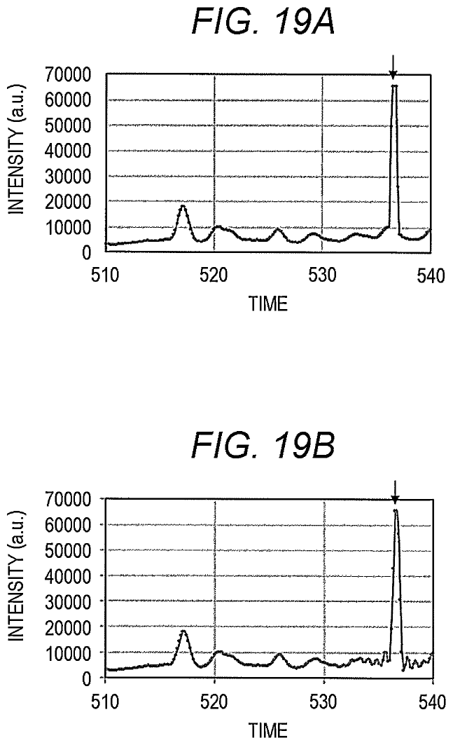

Here, a description will be given with reference to FIGS. 19A and 19B regarding a spike (the above-described specific spike) that is hardly determined as a spike by the magnitude of the absolute value of the peak height change rate after the correction.

An arrow in FIG. 19A indicates a spike before the data correction. A peak height is saturated at a measurement upper limit value, and is of three successive points. Regarding a spike after the data correction, a peak height is still saturated at the measurement upper limit and is of two successive points as illustrated in FIG. 19B. A change rate of the peak height by the data correction is 0.0%.

In this manner, it is difficult to determine a spike in which the peak height is saturated at the measurement upper limit value at a plurality of successive points as the spike based on the absolute value of the peak height change rate after the correction.

There is a possibility that such a spike in which the peak height is saturated at the measurement upper limit value can be determined by an existing determination method. Examples of the existing determination method include a method of performing determination based on a peak height before correction, a method of performing determination based on a half-value width of a peak before correction, a method of performing determination based on whether or not a peak before correction overlaps at a plurality of measurement wavelengths, a method of performing determination based on a range of a color in which the peak before correction appears, a combination thereof, and the like.

Therefore, if a method for discriminating between the sample-derived peak and the spike according to the present embodiment is used in combination with the existing determination method, more peaks can be correctly determined.

Although the example of the spike determination using the correction of the electrophoresis data has been described as above, the spike determination can be similarly performed even in the case of the post-color-call data.

FIG. 13A illustrates the post-color-call data obtained using the electrophoresis data that is not corrected, and FIG. 13C illustrates the post-color-call data obtained using the corrected electrophoresis data obtained by performing the correction on the same electrophoresis data. It can be seen that the peak height of the spike is reduced by the correction as compared with the sample-derived peak.

Further, FIG. 14A illustrates the post-color-call data in the case where correction is performed on the post-color-call data illustrated in FIG. 13A. In this case, it can be also seen that the peak height of the spike is reduced by the correction as compared with the sample-derived peak. Therefore, it is possible to discriminate between the sample-derived peak and the spike based on a difference in the change rate of the peak height caused by the correction.

As illustrated in FIGS. 18D, 18F, 18H, and 19B, a waveform that does not originally exist and corresponds to a cutoff frequency appears at the bottom of a corrected spike. Since the magnitude of the waveform appearing at the bottom depends on a peak height of the spike, the analysis is not affected if the spike is relatively small. However, if the peak height of the spike is higher as compared with a sample-derived peak, a shape and a size of the sample-derived peak are likely to change due to the waveform that does not originally exist but appears at the bottom of the spike, thereby changing an analysis result.

Therefore, the data correction may be performed after the spike is removed. Specifically, the correction is performed on electrophoresis data or post-color-call data as described in the first to fourth embodiments. Next, a spike is determined by the method in the fifth embodiment based on the peak intensities before and after correction and a conventional spike determination method.

Those skilled in the art can appropriately determine an adjustment method in a case where a determination result obtained by the method in the fifth embodiment and a determination result obtained by the conventional spike determination method do not match. For example, a peak determined as a spike by either method may be determined to be the spike, or only a peak determined as a spike by both the methods may be determined to be the spike.

Then, the spike is removed from the electrophoresis data or post-color-call data before correction, and the electrophoresis data or post-color-call data from which the spike has been removed is corrected again. As a result, it is possible to prevent the waveform that does not originally exist and corresponds to the cutoff frequency from appearing at the bottom of the corrected spike.

There are various methods as a method of removing a spike. For example, there is a method of removing a plot forming a spike, and then complementing a data point by nonlinear curve fitting or nonlinear peak fitting using data points around the removed plot.

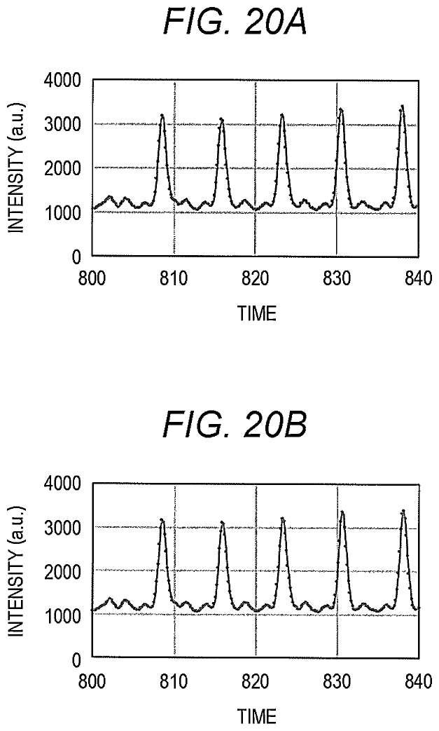

A process of removing a spike from electrophoresis data including the spike, complementing a data point by nonlinear curve fitting, and then, performing correction will be described using the following example. The data illustrated in FIG. 18G is used as the electrophoresis data including the spike. FIG. 20A illustrates a waveform of electrophoresis data obtained by removing the spike and complementing the data point. FIG. 20B illustrates a waveform of electrophoresis data obtained by performing data correction on the waveform of the electrophoresis data in FIG. 20A. In a case of comparing the waveform of FIG. 20B and the waveform of FIG. 18H obtained by performing the data correction without removing the spike, the disturbance of the waveform near the bottom of the spike that can be confirmed in FIG. 18H does not occur in FIG. 20B.

In this manner, the sample-derived peak and the spike can be more appropriately identified according to the fifth embodiment. As a result, the spike can be more easily removed, so that the noise included in the electrophoresis data can be further reduced, and the increase in sensitivity or the increase in dynamic range can be achieved.

What is claimed is:

1. A method for correcting data related to electrophoresis by removing a part of a noise component from the data, the method comprising:
acquiring first data by performing electrophoresis of a labeled nucleic acid sample to be analyzed and simultaneously detecting label signals at a plurality of measurement wavelengths, the first data being detection intensity waveform data containing a sample-derived component and a noise component;
selecting, from the first data, specific wavelength data corresponding to one or more measurement wavelengths which is a target of time-frequency analysis;
performing filtering processing to cut some or all of components on a high frequency side on the specific wavelength data for one or more cutoff frequencies;
comparing peak intensities of the specific wavelength data before and after the filtering processing for each of the cutoff frequencies;
calculating, as a first cutoff frequency, a minimum cutoff frequency at which a decrease in the peak intensity of the specific wavelength data falls within a predetermined allowable range among the cutoff frequencies; and
correcting the first data or post-color-call data of the first data by performing filtering processing with the first cutoff frequency.

2. The method according to claim 1, wherein the predetermined allowable range is 1% or less.

3. The method according to claim 1, further comprising acquiring a background value of the specific wavelength data,
wherein the peak intensity is represented using a difference between a peak top value and the background value.

4. The method according to claim 1, further comprising acquiring a background value of the specific wavelength data,
wherein the peak intensity is represented using an area of a peak.

5. The method according to claim 1, wherein
the specific wavelength data is data of a measurement wavelength determined to have no spike based on a predetermined criterion, or data of a measurement wavelength in which a peak value of a spike is determined to fall within a same range as a peak value of the sample-derived component based on a predetermined criterion.

6. The method according to claim 1, further comprising:
acquiring, as an initial cutoff frequency, a maximum frequency at which power of the sample-derived component is higher than power of a white noise level in a power spectrum of the specific wavelength data; and
calculating the first cutoff frequency by using the initial cutoff frequency as an initial value of the cutoff frequency and repeating the filtering processing while lowering the cutoff frequency.

7. The method according to claim 6, further comprising performing smoothing processing on the power spectrum upon acquiring the initial cutoff frequency.

8. The method according to claim 1, wherein
the first data is measurement value data obtained by electrophoresis, and
the correcting is correcting the first data by performing the filtering processing with the first cutoff frequency.

9. The method according to claim 1, wherein
the first data is measurement value data obtained by electrophoresis, and
the correcting is correcting the post-color-call data of the first data by performing the filtering processing with the first cutoff frequency.

10. The method according to claim 1, wherein
the first data is post-color-call data of measurement value data obtained by electrophoresis, and
the correcting is correcting the first data by performing the filtering processing with the first cutoff frequency.

11. A method for determining whether each of peaks in data related to electrophoresis is a sample-derived peak or a spike, the method comprising:
performing correction using the method according to claim 1;
calculating a peak intensity change rate for each of the peaks based on a peak intensity before the correction and a peak intensity after the correction; and
determining that the peak at which an absolute value of the peak intensity change rate is greater than a predetermined threshold at one or more measurement wavelengths is the spike.

12. The method according to claim 11, wherein the predetermined threshold exceeds an upper limit of the predetermined allowable range.

13. The method according to claim 12, wherein the predetermined threshold is equal to or greater than twice the upper limit of the predetermined allowable range.

14. An apparatus configured to execute the method according to claim 1.

15. A program for causing a computer to execute the method according to claim 1.
